(19) United States
(12) Patent Application Publication
(10) Pub. No.: US 2020/0188449 A1
(43) Pub. Date: Jun. 18, 2020

(54) Compositions and Methods for Treating Chronic Fatigue Syndrome and Related Disorders

(71) Applicant: Crestovo Holdings LLC, Somerville, MA (US)

(72) Inventor: Thomas Julius BORODY, Five Dock (AU)

(73) Assignee: Crestovo Holdings LLC, Somerville, MA (US)

(21) Appl. No.: 16/341,164

(22) PCT Filed: Oct. 11, 2017
(86) PCT No.: PCT/US2017/056126

§ 371 Date: Apr. 11, 2019

Related U.S. Application Data
(60) Provisional application No. 62/406,756, filed on Oct. 11, 2016.
(60) Provisional application No. 62/511,790, filed on May 26, 2017.

(51) Int. Cl. A61K 35/74; A61K 35/38; A61P 25/00

(52) U.S. Cl. CPC A61K 35/74; A61K 9/0053; A61P 25/00; A61K 35/38

(57) ABSTRACT

The present disclosure is in the field of pharmaceutical compositions suitable for the treatment of diseases in mammals. The disclosure provides novel compositions comprising non-pathogenic fecal microbes for treating chronic fatigue syndrome and related diseases. The disclosure also provides methods for treating a subject with the compositions disclosed herein.

### CROSS-REFERENCE TO RELATED APPLICATIONS

This application claims priority to U.S. Provisional Application No. 62/406,756, filed Oct. 11, 2016, and U.S. Provisional Application No. 62/511,790, filed May 26, 2017. Each of these U.S. Provisional Applications are incorporated by reference herein in their entireties.

### FIELD

The present disclosure relates to pharmaceutical compositions and methods suitable for treating gastrointestinal (GI) disorders, such as chronic fatigue syndrome.

### BACKGROUND

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and results in increased susceptibility to pathogens, altered metabolic profiles, or induction of proinflammatory signals that can lead to local or systemic inflammation or autoimmunity. Additionally, such a disrupted microbiota may be infected by incoming pathogen or pathogens, which can cause pain, diarrhea, gas, and constipation among other symptoms. Hence, the intestinal microbiota plays a significant role in the pathogenesis of many disorders such as pathogenic infections of the gut.

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE). It entails infusions through a colonoscope, an enema or via a nasojejunal tube of human microbiota either in the form of homogenised stool, or cultured stool components such as Clostridia, to implant in the colon and thereby displace or eradicate pathogenic bacteria, e.g., C. difficile.

Chronic fatigue syndrome (CFS) is a debilitating and complex disorder characterized by extreme fatigue that is not improved by bed rest. This disorder can worsen with ongoing mental and physical activity. There is no cure for chronic fatigue syndrome and no prescription drugs have been developed specifically for this disease to date. Thus, there is a need for treatments for chronic fatigue syndrome.

### SUMMARY

The present disclosure provides compositions, methods, and dosing regimens for treating or preventing chronic fatigue syndrome.

In one aspect, the present disclosure provides a method for treating chronic fatigue syndrome in a subject in need thereof, where the method comprises administering to the subject a pharmaceutically active dose of a therapeutic composition comprising or derived from live non-pathogenic fecal bacteria or a sterile fecal filtrate. In one aspect, a sterile fecal filtrate originates from a donor stool. In another aspect, a sterile fecal filtrate originates from cultured microorganisms.

In another aspect, this disclosure provides use of a composition comprising live non-pathogenic fecal bacteria in the manufacture of a medication for the treatment of chronic fatigue syndrome.

In one aspect, a method provided here is for treating a fatigue condition selected from the group consisting of chronic idiopathic fatigue (CIF) and CFS-like with insufficient fatigue syndrome (CFSLWIFS).

In one aspect, a treatment method achieves at least 50% reduction of the Sum8 ancillary index in at least 50% patients after 8 weeks of treatment compared to baseline (e.g., immediately prior to treatment).

In one aspect, the present disclosure provides a method for treating chronic fatigue syndrome in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily or at least once or twice weekly for at least three, eight, ten, or twenty consecutive weeks. In a further aspect, the dose is administered at a dosing schedule of at least once or twice daily or at least one or twice weekly for at least four, five, six, seven, eleven, twelve, thirteen, fourteen, fifteen, sixteen, seventeen, eighteen, or nineteen consecutive weeks.

In one aspect, the present disclosure provides a method for treating chronic fatigue syndrome in a subject in need thereof, where the method comprises administering orally to the subject a pharmaceutically active dose of a therapeutic composition comprising a liquid, frozen, lyophilized, or encapsulated sterile fecal filtrate, where the dose is administered at a dosing schedule of at least once or twice daily or at least once or twice weekly for at least three, eight, ten, or twenty consecutive weeks.

In one aspect, a method achieves a remission, cure, response, or resolution rate of chronic fatigue syndrome of at least about 80%.

In an aspect, a fecal microbiota in a therapeutic composition comprises a donor's substantially entire and non-selected fecal microbiota, reconstituted fecal material, or synthetic fecal material.

### BRIEF DESCRIPTION OF THE DRAWINGS

FIG. 1 shows response to initial and second transcolonoscopic infusion in CFS patients in accordance with Example 2 of the present disclosure;

FIG. 2 shows resolution of gastrointestinal symptoms in CFS response compared to non-response group in accordance with Example 2 of the present disclosure;

FIG. 3 shows the number of patients who remained CFS-free at 15-20 year follow-up in accordance with Example 2 of the present disclosure;

FIG. 4 shows response rate to therapy in sub-groups of CFS patients in accordance with Example 2 of the present disclosure.

### DETAILED DESCRIPTION

Unless defined otherwise herein, terms are to be understood according to conventional usage by those of ordinary skill in the relevant art.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, “microbiota,” and “flora” refer to a community of microbes that live in or on a subject's body, both sustainably and transiently, including eukaryotes, archaea, bacteria, and viruses (including bacterial viruses (i.e., phage)). A non-selected fecal microbiota refers to a community or mixture of fecal microbes derived from a donor's fecal sample without selection and substantially resembling microbial constituents and population structure found in such fecal sample.

As used herein, a “sterile fecal filtrate” or a “non-cellular fecal filtrate” refers to a liquid component of a fecal material, where the liquid component is free or substantially free of cell-based living organisms (e.g., bacteria, fungi, or their spores), but retains bacteriophages and non-cellular biological materials. Preferably, a non-cellular or sterile fecal filtrate is also free of viruses for eukaryotic host cells.

As used herein, “remission, cure, or resolution rate” refers to the percentage of patients that are cured or obtain remission or complete resolution of a condition in response to a given treatment. Remission, cure, or resolution of ulcerative colitis refers to complete cessation of fatigue and ancillary symptoms. Quantitatively, remission, cure, or resolution is achieved when a patient's Chronic Fatigue Syndrome severity score is below 3 and Sum8 score is below or equal to 14, assessed after 8 weeks of treatment. Remission, cure, or resolution can be further confirmed by functional Magnetic resonance imaging (fMRI) and proteomic analysis.

As used herein, “response rate” refers to the percentage of patients that respond positively (e.g., reduced severity or frequency of one or more symptoms) to a given treatment. Quantitatively, a patient responds to a treatment positively when the patient's Sum8 score decreases by at least 2 from baseline to week 8.

As used herein, “Chronic Fatigue Syndrome Severity Score” or “fatigue severity score” refers to an index system for assessing the symptomatic severity or response of a chronic fatigue syndrome patient. See Baraniuk et al., A Chronic Fatigue Syndrome (CFS) severity score based on case designation criteria. Am. J. Transl. Res. 2013; 5(1):53-68. The index is a subjective evaluation of the patient's severity of fatigue experienced in the previous 6 months. A score of 0 would be assigned to no symptoms, 1 for trivial, 2 for mild, 3 for moderate, and 4 for severe. This index is used in conjunction with “Sum8” ancillary scoring, which assesses eight variables, including problems with memory or concentration, sore throat, tender lymph node regions, myalgia, arthralgia, headaches, sleep disturbances, and exertional exhaustion. See Baraniuk et al. Each variable is scored from 0-4 so that the total index score ranges from 0-32. A Sum8≥14 accompanied with a fatigue severity score of 3 or 4 indicates that the subject is experiencing chronic fatigue syndrome. A sum8≤14 accompanied with a fatigue severity score of 3 or 4 indicates that the subject is experiencing chronic idiopathic fatigue. A sum8≥14 accompanied with a fatigue severity score of 0, 1, or 2 indicates that the subject is experiencing CFS-like with insufficient fatigue syndrome.

TABLE 1
Sum8 Index for chronic fatigue syndrome. See Baraniuk et al.

| | score |
|---|---|
| | assignment |
| 1. Problems with memory or | |
| concentration | |
| No Symptoms | 0 |
| Trivial | 1 |
| Mild | 2 |
| Moderate | 3 |
| Severe | 4 |
| 2. Sore Throat | |
| No Symptoms | 0 |
| Trivial | 1 |
| Mild | 2 |
| Moderate | 3 |
| Severe | 4 |
| 3. Tender Lymph Node Regions | |
| No Symptoms | 0 |
| Trivial | 1 |
| Mild | 2 |
| Moderate | 3 |
| Severe | 4 |
| 4. Myalgia | |
| No Symptoms | 0 |
| Trivial | 1 |
| Mild | 2 |
| Moderate | 3 |
| Severe | 4 |
| 5. Arthralgia | |
| No Symptoms | 0 |
| Trivial | 1 |
| Mild | 2 |
| Moderate | 3 |
| Severe | 4 |
| 6. Headaches | |
| No Symptoms | 0 |
| Trivial | 1 |
| Mild | 2 |
| Moderate | 3 |
| Severe | 4 |
| 7. Sleep Disturbances | |
| No Symptoms | 0 |
| Trivial | 1 |
| Mild | 2 |
| Moderate | 3 |
| Severe | 4 |
| 8. Exertional Exhaustion | |
| No Symptoms | 0 |
| Trivial | 1 |
| Mild | 2 |
| Moderate | 3 |
| Severe | 4 |

As used herein, “eukaryotic” refers to belonging to a cell that contains a nucleus and membrane-bound organelles.

As used herein, “bacteria,” “bacterium,” and “archaea” refer to single-celled prokaryotes that lack membrane bound nuclei and lack organelles.

As used herein, “fecal bacteria” refers to bacteria that can be found in fecal matter.

As used herein, “colony forming units” (cfu) refers to an estimate of the number of viable microorganism cells in a given sample.

As used herein, “viable” means possessing the ability to multiply.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula

$$H = - {\sum }_{i = 1}^{R} {p}_{i} ln {p}_{i}$$

where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” means that that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, a “continuous dosing schedule” refers to a dosing schedule where a therapeutic composition is administered during a treatment period without a rest period. Throughout the treatment period of a continuous dosing schedule, a therapeutic composition can be administered, for example, daily, or every other day, or every third day, or weekly. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time, for example, once per day, once a week, or once in two weeks.

As used herein, “dosing interval” refers to the amount of time that elapses between multiple doses being administered to a subject.

Chronic fatigue syndrome is a disease that is characterized by severe fatigue for six or more consecutive months that is not due to ongoing exertion or other medical conditions associated with fatigue. Such fatigue is not improved by bedrest and significantly interferes with daily activities and work. Chronic fatigue syndrome differs from the other fatigue conditions in that an individual diagnosed with the syndrome concurrently has 4 or more of the following symptoms: post-exertion malaise lasting more than 24 hours; unrefreshing sleep; significant impairment of short-term memory or concentration; muscle pain; multi-joint pain without swelling or redness; headaches of a new type, pattern, or severity; tender cervical or axillary lymph nodes; a sore throat that is frequent or recurring.

The cause of chronic fatigue syndrome is unknown. Healthcare practitioners identified that virus infection, hypotention, weakened immune system, and hormonal imbalances as possible contributing factors that causes the syndrome. The Centers for Disease Control and Prevention (CDC) suggests that chronic fatigue syndrome is the end stage of multiple different conditions, including Epstein-Barr virus infection and Ross River virus infection. Because many conditions have symptoms similar to chronic fatigue syndrome, such as mononucleosis, Lyme disease, multiple sclerosis, lupus, hypothyroidism, fibromyalgia, and major depressive disorder. Hence, a physician must rule out these clinical conditions before diagnosing that a patient has chronic fatigue syndrome.

Chronic fatigue syndrome occurs most often in people ages 40 to 60, although the disease may afflict people of any age. It affects women four times more frequently than men and appears to run in some families.

Other fatigue conditions exist. As used herein, “chronic idiopathic fatigue” or “CIF” refers to a condition where a subject experiences six or more consecutive months of severe fatigue, but experiences less than 4 of the symptoms listed in [0043]. As used herein, “CFS-like with insufficient fatigue syndrome” or “CFSLWIFS” refers to a condition where a subject experiences low fatigue while experiencing 4 or more of the symptoms listed in [0043].

Several theories have been proposed for the cause of chronic fatigue syndrome, yet no single cause have been identified. There is some evidence to suggest that certain infections may cause or trigger chronic fatigue syndrome. However, no single infection or pathogen has been identified to cause chronic fatigue syndrome. Some studies suggest that changes in an individual's immune system upon infection may lead to chronic production of cytokines, resulting in chronic fatigue syndrome. Altered hormonal balance (e.g. corticotrophin-releasing hormone and cortisol) may also influence the immune system to cause chronic fatigue syndrome. There is also a study relating chronic fatigue syndrome with abdominal symptoms. See Berstad et al., Functional bowel symptoms, fibromyalgia and fatigue: A food-induced triad? Scand J. Gastroenterol. 2012; 47(8-9):914-19.

Chronic fatigue syndrome may cause long-term problems in an individual including feelings of anger, guilt, anxiety, isolation, and abandonment. Other quality of life issues may arise including a decrease of stamina that interferes with activities of daily life, memory and concentration problems that seriously hurt work or school performance, loss of independence, livelihood, and economic security, and alterations in relationships with family and friends. These problems can disappear when the chronic fatigue syndrome is treated effectively.

There is no cure for chronic fatigue syndrome. Treatment programs are generally individualized to the needs and symptoms experienced by the patient. No drug is available for treating chronic fatigue syndrome itself, and physicians are discouraged to use medications to treat the symptoms because of the patients' sensitivity to drugs in general. Some non-drug therapies may include acupuncture, gentle massage, deep breathing, relaxation therapy, or yoga to increase energy and decrease pain. Other coping strategies may include professional counselling, attending a support group, and changing employment condition to one that is favorable to support chronic fatigue syndrome individuals.

Many chronic diseases and disorders of the GI tract have chronic infection/infestation as their underlying pathological conditions (e.g., chronic fatigue syndrome). In one aspect, the present disclosure includes and relates to the use of a fecal microbiota, one or more microbial species therefrom, an active fragment or component therefrom for the treatment and/or prophylaxis of various disease states (e.g., chronic fatigue syndrome) related to the presence of ‘abnormal’ microflora in the GI tract. An active fragment of a bacterium can be any active molecule isolated from such bacteria by any known method for preparing/identifying active fragments of bacteria and proteins secreted from bacteria. Such methods include but are not limited to the following: sonication, osmotic shock, detergent lysis, high pressure, transfer appropriate DNA to other organisms, such as bacteria, plant or animal that is then used as a feed additive as described previously. In one aspect, an active fragment or component of a bacterium is selected from the group consisting of a mycolate or a derivative thereof, a polysaccharide, a lipoglycan, a small peptide, a thiopeptide, a protein, a nucleic acid molecule, a metabolite, a cell wall component, or any combination thereof. In one aspect, an active fragment is a protein or a secretion. In another aspect, an active fragment is a secreted protein.

In one aspect, the present disclosure provides a method for treating chronic fatigue syndrome in a subject in need thereof, where the method comprises administering to the subject a pharmaceutically active dose of a therapeutic composition comprising live non-pathogenic fecal bacteria. In another aspect, this disclosure provides use of a composition comprising live non-pathogenic fecal bacteria in the manufacture of a medication for the treatment of chronic fatigue syndrome.

In one aspect, a method provided here is for treating a fatigue condition selected from the group consisting of chronic idiopathic fatigue (CIF) and CFS-like with insufficient fatigue syndrome (CFSLWIFS). In one aspect, a therapeutic composition comprises an isolated or purified population of live non-pathogenic fecal bacteria. In one aspect, a therapeutic composition comprises a non-selected fecal microbiota. In another aspect, a therapeutic composition comprises a non-selected and substantially complete fecal microbiota. In another aspect, a therapeutic composition comprises a full-spectrum fecal microbiota. In one aspect, a method further comprises administering a 5-aminosalicylic acid agent, a corticosteroid, an immunosuppressant, or a combination thereof. In another aspect, a method further comprises administering 5-aminosalicylic acid or a derivative thereof, sulfasalazine or a derivative thereof, or a combination thereof.

In one aspect, the present disclosure provides a method which eliminates or reduces one or more chronic fatigue syndrome symptoms selected from the group consisting of post-exertion malaise lasting more than 24 hours; unrefreshing sleep; sleep deprivation; lethargy/fatigue; significant impairment of short-term memory or concentration; muscle pain; multi joint pain without swelling or redness; headaches of a new type, pattern, or severity; tender cervical or axillary lymph nodes; a sore throat that is frequent or recurring.

In one aspect, the present disclosure provides a method for treating chronic fatigue syndrome in a subject in need thereof, where the method comprises administering to the subject a pharmaceutically active dose of a therapeutic composition comprising live non-pathogenic bacteria. In one aspect, the present disclosure provides a method for treating chronic fatigue syndrome in a subject in need thereof, where the method comprises administering daily or weekly to the subject a pharmaceutically active dose of a therapeutic composition comprising live non-pathogenic fecal bacteria. In one aspect, a therapeutic composition is administered to an chronic fatigue syndrome patient in need thereof at least once daily for at least two consecutive days. In one aspect, a therapeutic composition is administered at least once daily or at least once weekly for at least 3, 4, 5, 6, 7, 8, 9, 10, 11, 12, 13, 14, or 15 consecutive days. In another aspect, a therapeutic composition is administered at least once daily or at least once weekly for at least 1, 2, 3, 4, 5, 6, 7, 8, 9, 10, 11, or 12 consecutive weeks. In one aspect, a therapeutic composition is administered at least once daily or at least once weekly for at most 4, 5, 6, 7, 8, 9, 10, 11, 12, 13, 14, 15, 16, 17, 18, 19, or 20 consecutive days or weeks. In another aspect, a therapeutic composition is administered at least once daily or at least once weekly for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an chronic fatigue syndrome patient in need thereof at least twice daily for at least two consecutive days. In one aspect, a therapeutic composition is administered at least twice daily or at least twice weekly for at least 3, 4, 5, 6, 7, 8, 9, 10, 11, 12, 13, 14, or 15 consecutive days. In another aspect, a therapeutic composition is administered at least twice daily or at least twice weekly for at least 1, 2, 3, 4, 5, 6, 7, 8, 9, 10, 11, or 12 consecutive weeks. In one aspect, a therapeutic composition is administered at least twice daily or at least twice weekly for at most 4, 5, 6, 7, 8, 9, 10, 11, 12, 13, 14, 15, 16, 17, 18, 19, or 20 consecutive days or weeks. In another aspect, a therapeutic composition is administered at least twice daily or at least twice weekly for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an chronic fatigue syndrome patient in need thereof at least three times daily for at least two consecutive days. In one aspect, a therapeutic composition is administered at least three times daily or at least three times weekly for at least 3, 4, 5, 6, 7, 8, 9, 10, 11, 12, 13, 14, or 15 consecutive days. In another aspect, a therapeutic composition is administered at least three times daily or at least three times weekly for at least 1, 2, 3, 4, 5, 6, 7, 8, 9, 10, 11, or 12 consecutive weeks. In one aspect, a therapeutic composition is administered at least three times daily or at least three times weekly for at most 4, 5, 6, 7, 8, 9, 10, 11, 12, 13, 14, 15, 16, 17, 18, 19, or 20 consecutive days or weeks. In another aspect, a therapeutic composition is administered at least three times daily or at least three times weekly for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating chronic fatigue syndrome in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily for at least three consecutive days or weeks. In another aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating chronic fatigue syndrome in a subject in need thereof, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, a subject being treated is a subject already with chronic fatigue syndrome. Administration of a disclosed therapeutic composition to clinically, asymptomatic human subject who is genetically predisposed or prone to chronic fatigue syndrome is also useful in preventing the onset of clinical symptoms of chronic fatigue syndrome. A human subject genetically predisposed or prone to chronic fatigue syndrome can be a human subject having a close family member or relative exhibiting or having suffered chronic fatigue syndrome. In another aspect, a subject being treated is a subject in which chronic fatigue syndrome is to be prevented. In another aspect, a subject being treated is predisposed or susceptible to chronic fatigue syndrome. In another aspect, a subject being treated is a subject diagnosed as having chronic fatigue syndrome. In one aspect, a subject being treated is a patient in need thereof. In another aspect, a patient being treated is immunocompromised.

In one aspect, a subject being treated is a human patient. In one aspect, a patient is a male patient. In one aspect, a patient is a female patient. In one aspect, a patient is a premuature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old old patient (>85 years).

In one aspect, a method comprises administering a therapeutic composition orally, by enema, or via rectal suppository. In one aspect, a therapeutic composition administered herein is formulated as an enteric coated (and/or acid-resistant) capsule or microcapsule, or formulated as part of or administered together with a food, a food additive, a dairy-based product, a soy-based product or a derivative thereof, a jelly, flavored liquid, ice block, ice-cream, or a yogurt. In another aspect, a therapeutic composition administered herein is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is lyophilized, pulverized and powdered. It may then be infused, dissolved such as in saline, as an enema. Alternatively the powder may be encapsulated as enteric-coated and/or acid-resistant capsules for oral administration. These capsules may take the form of enteric-coated and/or acid-resistant microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition administered herein is in a liquid, frozen, freeze-dried, spray-dried, foam-dried, lyophilized, or powder form. In a further aspect, a therapeutic composition administered herein is formulated as a delayed or gradual enteric release form. In another aspect, a therapeutic composition administered herein comprises an excipient, a saline, a buffer, a buffering agent, or a fluid-glucose-cellobiose agar (RGCA) media. In another aspect, a therapeutic composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a therapeutic composition administered herein further comprises an acid suppressant, an antacid, an H2 antagonist, a proton pump inhibitor or a combination thereof. In one aspect, a therapeutic composition administered herein substantially free of non-living matter. In another aspect, a therapeutic composition administered herein substantially free of acellular material selected from the group consisting of residual fiber, DNA, viral coat material, and non-viable material.

In one aspect, a therapeutic composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides.

In one aspect, a method further comprises pretreating a subject with an antibiotic composition prior to administering a therapeutic bacterial or microbiota composition. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In a further aspect, a method further comprises pretreating a subject with an anti-inflammatory drug prior to administration of a therapeutic bacterial or microbiota composition.

In one aspect, a method achieves a remission, cure, response, or resolution rate of chronic fatigue syndrome of at least about 10%, 15%, 20%, 25%, 30%, 35%, 40%, 45%, 50%, 55%, 60%, 65%, 70%, 75%, 80%, 85%, 90%, 95%, 97%, or 99%. In an aspect, a method achieves a remission, cure, response, or resolution rate of chronic fatigue syndrome of from about 10% to about 100%, such as from about 10% to about 99%, from about 15% to about 99%, from about 15% to about 97%, from about 20% to about 95%, from about 25% to about 90%, from about 30% to about 85%, from about 35% to about 80%, from about 40% to about 75%, from about 45% to about 70%, from about 50% to about 65%, or from about 55% to about 60%. In one aspect, a treatment method achieves a reduction of Sum8 score after 8 weeks of treatment by more than 2, 3, 4, 5, 6, 7, 8, 9, 10, 11, 12, 13, 14, 15, 16, 17, 18, 19, or 20. In an aspect, a treatment method achieves a reduction of Sum8 score after 8 weeks of treatment by 2 to 32, such as 2 to 15, 10 to 25, 20 to 32, 5 to 30, 10 to 25, or 15 to 20. In one aspect, a treatment method achieves a reduction of Sum8 score after 8 weeks of treatment by more than 2, 3, 4, 5, 6, 7, 8, 9, 10, 11, 12, 13, 14, 15, 16, 17, 18, 19, or 20 in at least 10%, 20%, 30%, 50%, 60%, 70%, 80%, or 90% patients in a patient population. In an aspect, a treatment method achieves a reduction of Sum8 score after 8 weeks of treatment by 2 to 32, such as 2 to 15, 10 to 25, 20 to 32, 5 to 30, 10 to 25, or 15 to 20 in at least 10%, 20%, 30%, 50%, 60%, 70%, 80%, or 90% patients in a patient population. In one aspect, a treatment method achieves a reduction of Sum8 score after 8 weeks of treatment by more than 2, 3, 4, 5, 6, 7, 8, 9, 10, 11, 12, 13, 14, 15, 16, 17, 18, 19, or 20 in 10% to 95% patients in a patient population, such as 10% to 90%, 15% to 95%, 20 to 90%, 30 to 80%, 40 to 70%, or 50 to 60%. In one aspect, a treatment method achieves at least 10%, 20%, 30%, 50%, 60%, 70%, 80%, or 90% reduction of Sum8 score after 8 weeks of treatment compared to baseline (e.g., immediately prior to treatment). In an aspect, a treatment method achieves at least 10%, 20%, 30%, 50%, 60%, 70%, 80%, or 90% reduction of Sum8 score after 8 weeks of treatment compared to baseline (e.g., immediately prior to treatment). In one aspect, a treatment method achieves at least 10%, 20%, 30%, 40%, 50%, 60%, 70%, 80%, or 90% in in at least 10%, 20%, 30%, 50%, 60%, 70%, 80%, or 90% patients after 8 weeks of treatment compared to baseline (e.g., immediately prior to treatment). In one aspect, a treatment method achieves 10% to 100% reduction of Sum8 score, such as 10% to 45%, 45% to 90%, 10% to 25%, 25% to 40%, 40% to 55%, 55% to 70%, 70% to 85%, 85% to 100%, 20% to 90%, 30% to 80%, 40% to 70%, or 50 to 60% in at least 10%, 20%, 30%, 50%, 60%, 70%, 80%, or 90% patients after 8 weeks of treatment compared to baseline (e.g., immediately prior to treatment). In one aspect, a treatment method achieves at least 10%, 20%, 30%, 40%, 50%, 60%, 70%, 80%, or 90% in at least 10% to 95% patients after 8 weeks of treatment compared to baseline (e.g., immediately prior to treatment), such as 10% to 90%, 15% to 95%, 20 to 90%, 30 to 80%, 40 to 70%, or 50 to 60%.

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², or 10¹³ cfu. In another aspect, a pharmaceutically active therapeutic effective dose comprises at most about 10⁵, 10⁶, 10⁷, 10⁸, 10⁹, 10¹⁰, 10¹¹, 10¹², 10¹³, 10¹⁴, or 10¹⁵ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², or 10¹³ cells or spores. In another aspect, a pharmaceutically active or therapeutic effective dose comprises at most about 10⁵, 10⁶, 10⁷, 10⁸, 10⁹, 10¹⁰, 10¹¹, 10¹², or 10¹³ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a therapeutic composition administered herein comprises fecal bacteria. In one aspect, a therapeutic composition administered herein comprises one or more, two or more, three or more, four or more, or five or more isolated, purified, or cultured microorganisms selected from the group consisting of Acinetobacter, Akkermansia, Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia. In one aspect, a therapeutic composition administered herein comprises one or more, two or more, three or more, four or more, or five or more isolated, purified, or cultured microorganisms selected from the group consisting of Acidaminococcus, Acinetobacter, Akkermansia, Alistipes, Anaerotruncus, Bacteroides, Bifidobacterium Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella.

In one aspect, a therapeutic composition administered herein comprises at least one, at least two, at least three, at least four, at least five, at least six, or at least seven fecal microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, Desuifomonas pigra, Lactobacillus G, Succinivibrio A, and a combination thereof.

In one aspect, a therapeutic composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a therapeutic composition administered herein comprises no viable Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, —CC; Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, Desuifomonas pigra, Lactobacillus G, Succinivibrio A, or a combination thereof.

In one aspect, a therapeutic composition administered herein comprises a fecal microbiota. In another aspect, the preparation of a fecal microbiota used herein involves a treatment selected from the group consisting of ethanol treatment, detergent treatment, heat treatment, irradiation, and sonication. In another aspect, the preparation of a fecal microbiota used herein involves no treatment selected from the group consisting of ethanol treatment, detergent treatment, heat treatment, irradiation, and sonication. In one aspect, the preparation of a fecal microbiota used herein involves a separation step selected from the group consisting of density gradients, filtration (e.g., sieves, nylon mesh), and chromatography. In another aspect, the preparation of a fecal microbiota used herein involves no separation step selected from the group consisting of density gradients, filtration (e.g., sieves, nylon mesh), and chromatography. In another aspect, a fecal microbiota used herein comprises a donor's entire fecal microbiota. In another aspect, a therapeutic composition administered herein comprises a fecal microbiota substantially free of eukaryotic cells from the fecal microbiota's donor.

In another aspect, a therapeutic composition administered herein comprises a fecal microbiota further supplemented, spiked, or enhanced with a fecal microorganism. In one aspect, a fecal microbiota is supplemented with a non-pathogenic (or with attenuated pathogenicity) bacterium of Acinetobacter, Akkermansia, Clostridium, Collinsella, Dorea, Ruminococcus, Coprococcus, Prevotella, Veillonella, Bacteroides, Baccillus, or a combination thereof. In another aspect, a therapeutic composition administered herein comprises a fecal microbiota further supplemented, spiked, or enhanced with a species of Acinetobacter, Akkermansia, Veillonellaceae, Firmicutes, Gammaproteobacteria, Bacteroidetes, or a combination thereof. In another aspect, a therapeutic composition administered herein comprises a fecal microbiota further supplemented with fecal bacterial spores. In one aspect, fecal bacterial spores are Clostridium spores, Bacillus spores, or both.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless.

In an aspect, a therapeutic composition provided or administered herein comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 3.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In another aspect, a therapeutic composition comprises fecal bacteria from at least 11, 12, 13, 14, 15, 16, 17, 18, 19, or 20 different families. In yet another aspect, a therapeutic composition comprises fecal bacteria from at least 21, 22, 23, 24, 25, 26, 27, 28, 29, or 30 different families. In a further aspect, a therapeutic composition comprises fecal bacteria from at least 31, 32, 33, 34, 35, 36, 37, 38, 39, or 40 different families. In another aspect, a therapeutic composition comprises fecal bacteria from at least 41, 42, 43, 44, 45, 46, 47, 48, 49, or 50 different families. In another aspect, a therapeutic composition comprises fecal bacteria from between 1 and 10, between 10 and 20, between 20 and 30, between 30 and 40, between 40 and 50 different families. In an aspect, a therapeutic composition provided or administered herein comprises a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided or administered herein comprises a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a therapeutic composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.542 mm, 0.5 mm, 0.446 mm, 0.33 mm, 0.381 mm, 0.295 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, 0.002 mm, 0.1 mm, or 0.2 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human. In an aspect, a therapeutic composition provided or administered herein comprises an extract of human feces where the composition is substantially odorless. In an aspect, a therapeutic composition provided or administered herein comprises fecal material or a fecal floral preparation in a lyophilized, crude, semi-purified or purified formulation.

In an aspect, a fecal microbiota in a therapeutic composition comprises highly refined or purified fecal flora, e.g., substantially free of non-floral fecal material. In an aspect, a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a fecal microbiota in a therapeutic composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a fecal microbiota in a therapeutic composition comprises a donor's substantially entire or non-selected fecal microbiota, reconstituted fecal material, or synthetic fecal material. In another aspect, the fecal microbiota in a therapeutic composition comprises no antibiotic resistant population. In another aspect, a therapeutic composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a fecal microbiota in a therapeutic composition used herein is derived from disease-screened fresh homologous feces or equivalent freeze-dried and reconstituted feces. In an aspect, a fresh homologous feces does not include an antibiotic resistant population. In another aspect, a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Acinetobacter, Akkermansia, Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a therapeutic composition used in a treatment disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911 (2017).

In one aspect, a fecal filtrate comprises secreted, execreted or otherwise liquid components or a microbiota, e.g., biologically active molecules (BAMs), which can be antibiotics or anti-inflammatories, are preserved, retained or reconstituted in a flora extract.

In one aspect, an exemplary therapeutic composition comprises starting material from a donor from a defined donor pool, where this donor contributes a stool that is centrifuged, then filtered with very high-level filtration using e.g., either metal sieving or Millipore filters, or equivalent, to ultimately permit only cells of bacterial origin to remain, e.g., often less than about 5 micrometers diameter. After the initial centrifugation, the solid material is separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation is now taken and filtered progressively in a filtering, e.g., a Millipore filtering or equivalent systems, to end up with liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a therapeutic composition used here comprises a reconstituted fecal flora consisting essentially of a combination of a purified fecal microbiota and a non-cellular fecal filtrate. In another aspect, a therapeutic composition used here comprises a purified fecal microbiota supplemented with one or more non-cellular non-particulate fecal components. In one aspect, a therapeutic composition used here comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a treatment method provided here comprises the use of both fecal bacterial cells, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a fecal bacteria-based therapeutic composition is used concurrently with a fecal non-cellular filtrate-based therapeutic composition. In another aspect, a patient is treated with a first fecal non-cellular filtrate-based therapeutic composition before being given a second fecal bacteria-based therapeutic composition, or vice versa. In a further aspect, a treatment method comprises three steps: first, antibiotic pre-treatment to non-selectively remove infectious pathogen(s); second, a fecal non-cellular filtrate-based treatment step to further suppress selected infectious pathogen(s); and third, giving the patient a fecal bacteria-based therapeutic composition to re-establish a functional intestinal microbiome.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of chronic fatigue syndrome. The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a therapeutic composition comprises at least about 10⁵, 10⁶, 10⁷, 10⁸, 10⁹, 10¹⁰, 10¹¹, 10¹², or 10¹³ cfu or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cfu or total cell count.

In another aspect, a therapeutic composition comprises at least about 10⁵, 10⁶, 10⁷, 10⁸, 10⁹, 10¹⁰, 10¹¹, 10¹², or 10¹³ cells or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cells or total cell count.

In one aspect, a therapeutic composition is formulated as an oral capsule, microcapsule, tablet, or pill. In another aspect, a capsule, microcapsule, tablet, or pill is adapted for enteric delivery. In a further aspect, a capsule, microcapsule, tablet, or pill is an enteric capsule, microcapsule, tablet, or pill. In another aspect, a capsule, microcapsule, tablet, or pill comprises an enteric coating, is acid resistant, or both.

In an aspect, this application provides for the following embodiments:

### Embodiment 1

A method for treating chronic fatigue syndrome in a subject in need thereof, said method comprising administering to said subject a pharmaceutically active dose of a therapeutic composition comprising live non-pathogenic fecal bacteria or a non-cellular fecal filtrate.
a. Embodiment 2: A method for treating a fatigue condition in a subject in need thereof, said method comprises administering to said subject a pharmaceutically active dose of a composition comprising live non-pathogenic fecal bacteria, wherein said fatigue condition is selected from the group consisting of chronic idiopathic fatigue (CIF) and CFS-like with insufficient fatigue syndrome (CFSLWIFS).
b. Embodiment 3: The method of embodiment 1 or 2, wherein said composition comprises an isolated or purified population of said live non-pathogenic fecal bacteria.
c. Embodiment 4: The method of embodiment 1 or 2, wherein said composition comprises a non-selected fecal microbiota.
d. Embodiment 5: The method of embodiment 1 or 2, wherein said method reduces the Sum8 score of said-subject by at least 10%, 20%, 30%, 50%, 60%, 70%, 80%, or 90% after 8 weeks of treatment.
e. Embodiment 6: The method of embodiment 1 or 2, wherein said administration is on a daily or weekly basis.
f. Embodiment 7: The method of embodiment 1 or 2, wherein said administration lasts at least 1, 2, 3, 4, 5, 6, 7, 8, 9, or 10 weeks.

### Embodiment 8

The method of embodiment 1 or 2, wherein said dose is administered at least once daily or weekly for at least 2, 3, 4, 5, 6, 7, 8, 9, 10, 11, 12, 13, 14, or 15 consecutive days or weeks.

### Embodiment 9

The method of embodiment 1 or 2, wherein said dose is administered at least once daily or weekly for at least 1, 2, 3, 4, 5, 6, 7, 8, 9, 10, 11, or 12 consecutive weeks.

### Embodiment 10

The method of embodiment 1 or 2, wherein said dose is administered at least once daily or weekly for at most 3, 4, 5, 6, 7, 8, 9, 10, 11, 12, 13, 14, 15, 16, 17, 18, 19, or 20 consecutive days or weeks.

### Embodiment 11

The method of embodiment 1 or 2, wherein said dose is administered at least once daily or weekly for at most 1, 2, 3, 4, 5, 6, 7, 8, 9, 10, 11, or 12 consecutive weeks.

### Embodiment 12

The method of embodiment 1 or 2, wherein said dose is administered at least twice daily for at least two consecutive days.

### Embodiment 13

The method of embodiment 12, wherein said dose is administered at least twice daily or weekly for at least 3, 4, 5, 6, 7, 8, 9, 10, 11, 12, 13, 14, or 15 consecutive days or weeks.

### Embodiment 14

The method of embodiment 12, wherein said dose is administered at least twice daily or weekly for at least 1, 2, 3, 4, 5, 6, 7, 8, 9, 10, 11, or 12 consecutive weeks.

### Embodiment 15

The method of embodiment 12, wherein said dose is administered at least twice daily or weekly for at most 3, 4, 5, 6, 7, 8, 9, 10, 11, 12, 13, 14, 15, 16, 17, 18, 19, or 20 consecutive days or weeks.

### Embodiment 16

The method of embodiment 12, wherein said dose is administered at least twice daily or weekly for at most 1, 2, 3, 4, 5, 6, 7, 8, 9, 10, 11, or 12 consecutive weeks.

### Embodiment 17

The method of embodiment 1 or 2, wherein said dose is administered at least three times daily for at least one day.

### Embodiment 18

The method of embodiment 17, wherein said dose is administered at least three times daily or weekly for at least 2, 3, 4, 5, 6, 7, 8, 9, 10, 11, 12, 13, 14, or 15 consecutive days or weeks.

### Embodiment 19

The method of embodiment 17, wherein said dose is administered at least three times daily or weekly for at most 2, 3, 4, 5, 6, 7, 8, 9, 10, 11, 12, 13, 14, or 15 consecutive days or weeks.

### Embodiment 20

The method of embodiment 1, wherein said therapeutic composition comprises both live non-pathogenic fecal bacteria and a non-cellular fecal filtrate.

### Embodiment 21

The method of embodiment 1, wherein therapeutic composition comprises live non-pathogenic fecal bacteria supplemented with a non-cellular fecal filtrate.

### Embodiment 22

The method of embodiment 1, 20, or 21, wherein said non-cellular fecal filtrate comprises biologically active proteins or peptides, micronutrients, fats, sugars, small carbohydrates, trace elements, mineral salts, ash, mucous, amino acids, nutrients, vitamins, minerals, or any combination thereof.

### Embodiment 23

The method of embodiment 1, 20, or 21, wherein said non-cellular fecal filtrate comprises one or more biologically active molecules selected from the group consisting of bacteriocin, lanbiotic, and lacticin.

### Embodiment 24

The method of embodiment 1, 20, or 21, wherein said non-cellular fecal filtrate comprises one or more bacteriocins selected from the group consisting of colicin, troudulixine, putaindicine, microcin, and subtilosin A.

### Embodiment 25

The method of embodiment 1, 20, or 21, wherein said non-cellular fecal filtrate comprises one or more lanbiotics selected from the group consisting of thuricin, nisin, subtilin, epidermin, mutacin, mersacidin, actagardine, and cinnamycin.

### Embodiment 26

The method of embodiment 1, 20, or 21, wherein said non-cellular fecal filtrate comprises an anti-spore compound, an antimicrobial compound, an anti-inflammatory compound, or any combination thereof.

### Embodiment 27

The method of embodiment 1, 20, or 21, wherein said non-cellular fecal filtrate comprises an interleukin, a cytokine, a leukotriene, an eicosanoid, or any combination thereof.

### Embodiment 28

The method of any one of preceding embodiments, wherein said method comprises a first dosing schedule followed by a second dosing schedule.

### Embodiment 29

The method of embodiment 28, wherein said second dosing schedule comprises a maintenance dose lower or equal to the dose of said first dosing schedule.

### Embodiment 30

The method of embodiment 29, wherein said second dosing schedule lasts for at least about 2, 4, 6, 8, 10, 12, 18, 24, 36, 48, 72, or 96 months.

### Embodiment 31

The method of embodiment 29, wherein said second dosing schedule lasts permanently.

### Embodiment 32

The method of embodiment 28, wherein the interval between said first and second dosing schedules is at least about 1, 2, 3, 4, 5, 6, 7, 8, 9, 10, 11, or 12 weeks.

### Embodiment 33

The method of embodiment 28, wherein said second dosing schedule is an continuous dosing schedule.

### Embodiment 34

The method of embodiment 28, wherein said second dosing schedule is an intermittent dosing schedule.

### Embodiment 35

The method of embodiment 34, wherein said intermittent dosing schedule comprises a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days.

### Embodiment 36

The method of any one of preceding embodiments, wherein said composition is formulated as a delayed or gradual enteric release form.

### Embodiment 37

The method of any one of preceding embodiments, wherein said administering comprises administering orally, by enema, or via rectal suppository.

### Embodiment 38

The method of any one of preceding embodiments, wherein said composition is formulated as an enteric coated capsule, an acid-resistant, enteric-coated capsule, an enteric coated microcapsule, or formulated as part of a food, a food additive, a dairy-based product, a soy-based product or a derivative thereof, a jelly, or a yogurt.

### Embodiment 39

The method of any one of preceding embodiments, wherein said method eliminates or reduces gastrointestinal dysbiosis.

### Embodiment 40

The method of any one of preceding embodiments, wherein said method increase bacterial diversity in said subject's gastrointestinal tract.

### Embodiment 41

The method of any one of preceding embodiments, wherein said pharmaceutically active dose comprises at least about 10⁵, 10⁶, 10⁷, 10⁸, 10⁹, 10¹⁰, 10¹¹, 10¹², or 10¹³ cfu or total number of cells.

### Embodiment 42

The method of embodiment 1 or 2, wherein said pharmaceutically active dose comprises at most about 10⁵, 10⁶, 10⁷, 10⁸, 10⁹, 10¹⁰, 10¹¹, 10¹², or 10¹³ cfu or total number of cells.

### Embodiment 43

The method of embodiment 1 or 2, wherein said pharmaceutically active dose is selected from the group consisting of from 10⁵ cfu to 10¹⁴ cfu, from 10⁶ cfu to 10¹⁴ cfu, from 10⁷ cfu to 10¹⁴ cfu, from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu or total number of cells.

### Embodiment 44

The method of embodiment 1 or 2, wherein said composition comprises a fecal microbiota further supplemented with a fecal microorganism.

### Embodiment 45

The method of embodiment 44, wherein said fecal microorganism is selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, —CC; Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, Desuifomonas pigra, Lactobacillus G, Succinivibrio A, Acinetobacter, Akkermansia, and a combination thereof.

### Embodiment 46

The method of embodiment 4, wherein said fecal microbiota is further supplemented with bacterial spores.

### Embodiment 47

The method of embodiment 46, wherein said bacterial spores are Clostridium spores or Bacillus spores.

### Embodiment 48

The method of embodiment 4, wherein the preparation of said fecal microbiota involves a treatment selected from the group consisting of ethanol treatment, detergent treatment, heat treatment, irradiation, and sonication.

### Embodiment 49

The method of embodiment 4, wherein the preparation of said fecal microbiota involves no treatment selected from the group consisting of ethanol treatment, detergent treatment, heat treatment, irradiation, and sonication.

### Embodiment 50

The method of embodiment 4, wherein the preparation of said fecal microbiota involves a separation step selected from the group consisting of density gradients, filtration, and chromatography.

### Embodiment 51

The method of embodiment 4, wherein the preparation of said fecal microbiota involves no separation step selected from the group consisting of density gradients, filtration, and chromatography.

### Embodiment 52

The method of embodiment 4, wherein said fecal microbiota comprises a donor's entire fecal microbiota.

### Embodiment 53

The method of embodiment 4, wherein said composition is substantially free of eukaryotic cells from said fecal microbiota's donor.

### Embodiment 54

The method of embodiment 4, wherein said fecal microbiota is from reconstituted fecal material.

### Embodiment 55

The method of embodiment 4, wherein said fecal microbiota is from synthetic fecal material.

### Embodiment 56

The method of embodiment 4, wherein said fecal microbiota comprises no antibiotic resistant population.

### Embodiment 57

The method of embodiment 4, wherein said fecal microbiota comprises a preparation of viable flora in proportional content that resembles a normal healthy human fecal flora.

### Embodiment 58

The method of embodiment 4, wherein said fecal microbiota comprises bacteria from at least seven different families.

### Embodiment 59

The method of embodiment 4, wherein said fecal microbiota has a Shannon Diversity Index of 0.4-5.0.

### Embodiment 60

The method of embodiment 4, wherein said fecal microbiota comprises one or more microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, and Monilia.

### Embodiment 61

The method of embodiment 4, wherein said fecal microbiota comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof.

### Embodiment 62

The method of embodiment 4, wherein said fecal microbiota comprises one or more microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, —CC; Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, Desuifomonas pigra, Lactobacillus G, Succinivibrio A, Acinetobacter, Akkermansia, and a combination thereof.

### Embodiment 63

The method of embodiment 1 or 2, wherein said composition comprises at least about 20%, 30%, 40%, 50%, 60%, 70%, 80%, 85%, 90%, 95%, 99%, or 99.5% bacterial spores.

### Embodiment 64

The method of embodiment 1 or 2, wherein said composition is in a liquid, frozen, freeze-dried, spray-dried, foam-dried, lyophilized, or powder form.

### Embodiment 65

The method of embodiment 1 or 2, wherein said composition comprises an excipient, a saline, a buffer, a buffering agent, or a fluid-glucose-cellobiose agar (RGCA) media.

### Embodiment 66

The method of embodiment 1 or 2, wherein said composition comprises a cryoprotectant.

### Embodiment 67

The method of embodiment 66, wherein said cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

### Embodiment 68

The method of embodiment 1 or 2, wherein said composition further comprises an acid suppressant, an antacid, an H₂ antagonist, a proton pump inhibitor or a combination thereof.

### Embodiment 69

The method of embodiment 1 or 2, wherein said composition is substantially free of non-living matter.

### Embodiment 70

The method of embodiment 1 or 2, wherein said composition is substantially free of acellular material selected from the group consisting of residual fiber, DNA, viral coat material, and non-viable material.

### Embodiment 71

The method of embodiment 1 or 2, wherein said composition is formulated as an enteric coated capsule or microcapsule, an acid-resistant capsule or microcapsule, a powder suitable for reconstitution, a naso-duodenal infusion, or for delivery in the form of an enema or a colonoscopic infusion.

### Embodiment 72

The method of embodiment 1 or 2, wherein said composition is administered together with a food, a liquid beverage, a food additive, a dairy-based product, a soy-based product or a derivative thereof, a jelly, or a yogurt.

### Embodiment 73

The method of embodiment 1 or 2, wherein said subject is pretreated with an antibiotic prior to administration of said composition.

### Embodiment 74

The method of embodiment 73, wherein said antibiotic is selected from the group consisting of rifabutin, clarithromycin, clofazimine, vancomycin, rifampicin, nitroimidazole, chloramphenicol, and a combination thereof.

### Embodiment 75

The method of embodiment 73, wherein said antibiotic is selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.

### Embodiment 76

The method of any one of preceding embodiments, wherein said subject is pretreated with an anti-inflammatory drug prior to administration of said composition.

### Embodiment 77

The method of any one of preceding embodiments, wherein said composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

### Embodiment 78

The method of embodiment 1 or 2, wherein said composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus.

### Embodiment 79

The method of embodiment 1 or 2, wherein said composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

### Embodiment 80

The method of embodiment 78, wherein said composition comprises two or more genera selected from the group consisting of Collinsella, Coprococcus, Dorea, Eubacterium, and Ruminococcus.

### Embodiment 81

The method of embodiment 78, wherein said composition comprises two or more genera selected from the group consisting of Coprococcus, Dorea, Eubacterium, and Ruminococcus.

### Embodiment 82

The method of embodiment 78 or 79, wherein said plurality of viable non-pathogenic microorganisms comprise one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

### Embodiment 83

The method of any one of preceding embodiments, wherein said method eliminates or reduces one or more chronic fatigue syndrome symptoms selected from the group consisting of post-exertion malaise lasting more than 24 hours; unrefreshing sleep; sleep deprivation; lethargy or fatigue; significant impairment of short-term memory or concentration; muscle pain; multi-joint pain without swelling or redness; headaches of a new type, pattern, or severity; tender cervical or axillary lymph nodes; a sore throat that is frequent or recurring.

### Embodiment 84

The method of any one of preceding embodiments, wherein said method further comprises administering a 5-aminosalicylic acid agent, a corticosteroid, an immunosuppressant, or a combination thereof.

### Embodiment 85

The method of any one of preceding embodiments, wherein said method further comprises administering 5-aminosalicylic acid or a derivative thereof, Sulfasalazine or a derivative thereof, or a combination thereof.

The disclosure may be better understood by reference to the following non-limiting Examples, which are provided as exemplary of the disclosure. The following examples are presented in order to more fully illustrate the preferred aspects of the disclosure and should in no way be construed, however, as limiting the broad scope of the disclosure. Therefore, the scope of the appended claims should not be limited to the description of the aspects contained herein.

### EXAMPLES

### Example 1. Preparation of Fecal Microbiota

Fecal microbiota is prepared essentially according to protocols published in US2014/0147417 or WO2014/152484. Summarized below is an exemplary protocol.

Potential fecal microbiota donors are screened according to a list of criteria used to exclude unsuitable donors. Potential fecal microbiota donors are excluded if they have received antibiotics, laxatives, diet pills, immunomodulators or chemotherapy in the preceding three months. Potential fecal microbiota donors are excluded if they have a history of all known infectious diseases, morbid obesity, diabetes, irritable bowel syndrome, inflammatory bowel disease, chronic diarrhea, constipation, colorectal polyps or cancer, a compromised immune system, metabolic syndromes, chronic fatigue syndrome, major GI surgery, or other diseases or conditions potentially associated with specific changes in fecal microbiota. Potential fecal microbiota donors are excluded if they exhibit positive laboratory tests for C-reactive protein, erythrocyte sedimentation rate, hepatitis A, hepatitis B, hepatitis C, human immunodeficiency virus, human T-lymphotropic virus or syphilis. Potential fecal microbiota donors are excluded if they exhibit a positive test for stool ova, parasites or viruses. Potential fecal microbiota donors are excluded if they engage in high-risk sexual behaviors, have been incarcerated, or received any tattoos or body piercings in areas that have had disease epidemics within the past three months.

Donor fecal material (fresh feces) is collected in a sterilized container, and then it is transferred to a blender. Approximately 500-1000 mL 0.9% saline solution is added to the blender and thoroughly mixed with the fecal sample. The resulting suspension is filtered at least 4 times through strainers prior to collecting a final suspension. The final suspension is centrifuged in 50 mL tubes at 1200×g for 3 minutes. The supernatant is discarded and the pellet is gently resuspended in approximately 50 mL of sterile 0.9% saline solution. The centrifugation and resuspension steps are repeated 2 to 4 additional times. Upon the final centrifugation, the supernatant is discarded. If the fecal microbiota is to be used immediately, the resultant pellet is resuspended in 1.5-volumes of 0.9% saline solution by gently mixing. If the fecal microbiota is to be stored, the resultant pellet is resuspended in 10% sterile glycerol and stored at −80 degrees Centigrade. If fecal microbiota are frozen, they are warmed to room temperature prior to administration to a patient

### Example 2. Treatment of Chronic Fatigue Syndrome

A total of 60 patients presented with chronic fatigue syndrome (52 CFS patients with irritable bowel syndrome; 4 CFS patients with constipation; and 4 CFS-only patients) are recruited in a study. Five out of 60 patients receive a single transcolonoscopic (TC) infusion of 300 cc culture comprising 13 non-pathogenic enteric bacteria (Bacteriodetes, Clostridia, and E. coli). Fifty-two out of 60 patients undergo a two-day infusion (TC and rectal infusion), while 3 out of 60 patients undergo a three-day rectal infusion schedule (TC and two-day rectal infusion). Response/non-response to the infusion treatment is defined as a resolution or return of CFS symptoms including sleep deprivation and lethargy/fatigue at 4 week after the treatment.

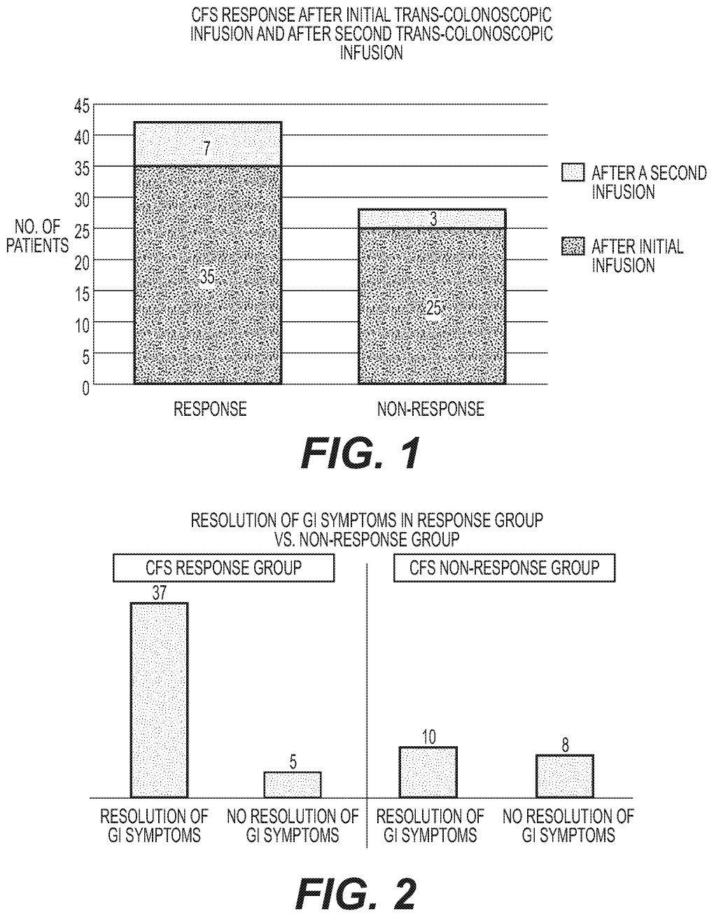

Thirty-five out of 60 patients who undergo initial bacteriotherapy respond to treatment. See FIG. 1. Ten out of 15 patients who fail to respond to the initial bacteriotherapy are offered a second TC infusion and either a follow-up rectal infusion (n=4) or an oral course of cultured bacteria (n=6). Referring to FIG. 1, 7 out of 10 respond to the follow-up dose. Accordingly, 42 out of 60 patients achieve clinical response to bacteriotherapy.

Referring to FIG. 2, of those patients who respond to bacteriotherapy, 37 out of 42 patients also experience resolution of their gastrointestinal symptoms. Of the 18 non-responders, 10 attain improvement of associated gastrointestinal symptoms despite persisting CFS symptoms.

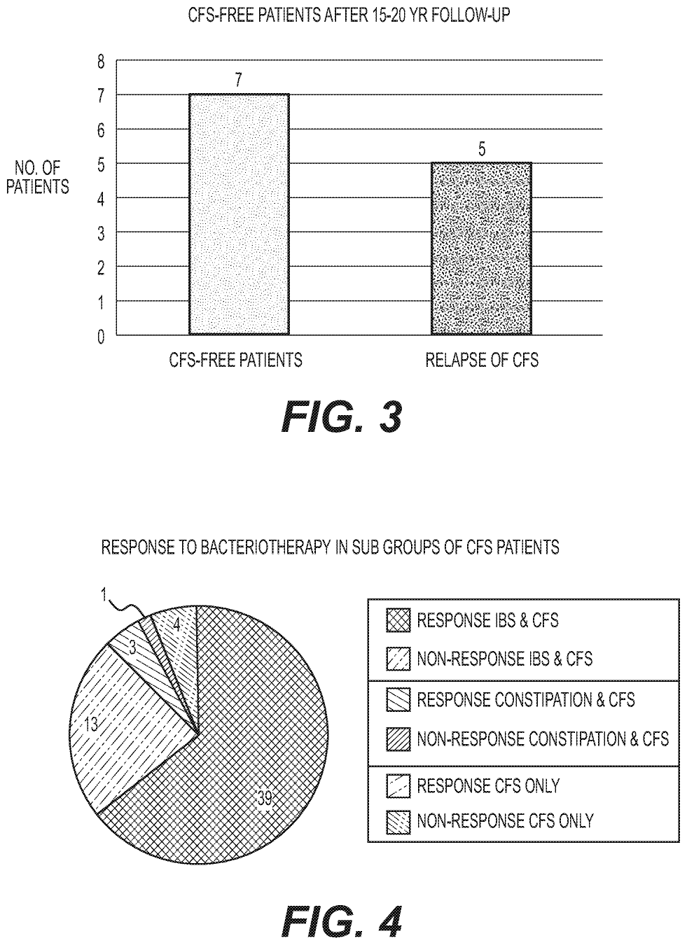

Referring to FIG. 3, at 15-20 year follow-up, 12 out of 60 patients are contactable and 7 out of 12 patients (58%) remain CFS-free. Five out of 12 patients experience CFS recurrence 1.5-3 years post the bacteriotherapy.

Referring to FIG. 4, bacteriotherapy is effective in those patients who have gastrointestinal conditions associated with CFS, and the treatment works to restore normal bacterial colonic concentrations. Namely, a bacterial pathophysiology is emphasized by the resolution of gastrointestinal symptoms in 78% of patients.

Therefore, bacteriotherapy achieves initial success rate of 70% in CFS and 58% sustained response. This result is favorable when compared with other therapies where fewer than 10% recover fully and a further 10%-20% worsen during follow-up. See Afrai and Buchwald. Am J. Psychiatry 2003; 160:221-36.

### Example 3. Oral Capsule Treatment Protocol for Chronic Fatigue Syndrome

Patients are divided into four groups (Groups 1 to 4). Group 1 patients are administered a pre-treatment of antibiotics (e.g., Vancomycin and Metronidazole) until diarrhea is controlled. Group 2 receives no antibiotics. Both Groups 1 and 2 receive a pre-colonoscopy bowel prep followed by capsule fecal microbiome therapy. Groups 3 and 4 receive no bowel prep while Group 3, not group 4, also receive an antibiotic pretreatment. Capsules are administered for 18 weeks as follows: two capsules twice-a-day for 14 days, two capsules twice-a-day every other day for 14 days, 4 capsules twice-a-week for 14 days, and 4 capsules once-a-week (e.g., each Monday) for 12 weeks. High dose capsules (total cell count of about 10¹²) are used in loading doses (also called treatment doses) for the initial 4 weeks. Lower dose capsules (total cell count of about 10⁹) are used in maintenance doses for the subsequent 14 weeks. In patients receiving antibiotic pretreatment, capsules are administered one day after ceasing antibiotics. Patient symptoms are observed and clinical examination is performed before, during and post oral capsule treatment. Pre, during and post-treatment DNA metagenomics (2-4 days; 1 week; 6 weeks; 12 weeks) are also carried out. The capsule treatments reverse patient symptoms and result in a clinically normal urge and defecation.

### Example 4. Treatment of a Patient with CFS

A 30-year old patient with CFS is treated with fecal microbiome therapy. A treatment regimen including acid resistant/delayed release double encapsulated oral capsules containing lyophilized donor-derived non-selected fecal microbiota is used. The patient's symptoms include moderate bloating, moderate abdominal discomfort, severe fatigue, severe abdominal pain, the feeling of pins and needles in legs, ringing in ears, and depression. The patient experiences 0-1 bowel movements per day. The patient is placed on a 6 week treatment protocol with 10 fresh fecal microbiome therapy liquid rectal enema infusions during the induction period of 2 weeks and 4 capsules (each containing a total cell count of about 10⁹-10¹²) per day for 4 weeks during the maintenance period. At week four post initial treatment, the patient experiences mild bloating, moderate abdominal discomfort, slight improvement in fatigue and cognitive issues, improvement in energy levels and vision. The patient also experiences a slight decrease in sharp abdominal pain and reports improvement in sleeping. At week 5 post initial treatment, the patient no longer experiences bloating and abdominal discomfort has improved to mild. Patient experiences improvement in energy levels, finds it easier to engage whilst participating in activities (e.g., watching TV, reading, and conversing with others). The patient's vision is blurry and cognitive issues are still present but vision and cognitive issues improve compared to before initial treatment. Patient has a 10-20% overall improvement at week 6 post initial treatment. Patient is flushed with Glyco and placed on a higher dose of capsules start at week 7. The patient is weaned off of fecal microbiome therapy as follows: week 7 includes 9 capsules/day; week 8 include 8 capsules/day; weeks 9 include 6 capsules/day; weeks 10-13 include maintenance dose of 4 capsules/day. At the end of week 13, patient has an approximate 40% improvement, has the ability to compose music, travel on band gigs, concentrate on verbal conversations, play light sports such as Frisbee, able to wake up clear-headed, able to observe quality sleep, able to limitedly socialize, brain fog still remains throughout the day, blurry visions comes and goes, still wakes up middle of the night to urinate 4 times/week, and overall has a sense of life returning to normal.

### Example 5. Treatment of a Patient with CFS

A 27-year old patient with CFS and C. perfingens is treated with fecal microbiome therapy. A treatment regimen including acid resistant/delayed release double encapsulated oral capsules containing lyophilized donor-derived non-selected fecal microbiota is used. The patient's symptoms include severe bloating, severe abdominal discomfort, severe urgency, severe fatigue, brain fog, flatulence, muscle aches, cramping, and inability to focus. The patient is placed on a 5 week treatment protocol with 5 fecal microbiome therapy liquid rectal enema infusions infusions during the induction period of 1 week and 2 capsules per day for 4 weeks during the maintenance period. As patient sees 10-20% improvement at the end of week 6 post-initial treatment, patient's capsule treatment is extended by 4 weeks. Patient's initial symptoms of fatigue decreases, able to attend work on a full time basis without having to leave work mid-day, brain fog has cleared, muscle aches have decreased and able to exercise more. Patient claims an overall 50-60% improvement from initial-treatment, however patient is still positive for C. perfingens.

As various modifications could be made in the constructions and methods herein described and illustrated without departing from the scope of the disclosure, it is intended that all matter contained in the foregoing description shall be interpreted as illustrative rather than limiting. The breadth and scope of the present disclosure should not be limited by any of the above-described exemplary embodiments, but should be defined only in accordance with the following claims appended hereto and their equivalents. All patent and non-patent documents cited in this specification are incorporated herein by reference in their entirety.

1. A method for treating chronic fatigue syndrome in a human subject in need thereof, said method comprising orally administering to said human subject a pharmaceutically active dose of a therapeutic composition comprising viable non-pathogenic bacteria of a fecal microbiota derived from a stool sample of a healthy human donor.

2. The method of claim 1, wherein said fatigue condition is selected from the group consisting of chronic idiopathic fatigue (CIF) and CFS-like with insufficient fatigue syndrome (CFSLWIFS).

3. (canceled)

4. The method of claim 1, wherein said composition comprises a substantially complete fecal microbiota of said stool sample.

5. (canceled)

6. The method of claim 1, wherein said method reduces the Sum8 score of said subject by at least 10%, 20%, 30%, 50%, 60%, 70%, 80%, or 90% after at least 8 weeks of treatment.

7. The method of claim 1, wherein said dose is administered at least once daily for at least 2 weeks.

8. The method of claim 1, wherein said dose is administered at least twice weekly for at least two weeks.

9. (canceled)

10. (canceled)

11. (canceled)

12. (canceled)

13. The method of claim 1, wherein said method comprises a first dosing schedule followed by a second dosing schedule, wherein said second dosing schedule comprises a maintenance dose lower or equal to the dose of said first dosing schedule.

14. The method of claim 1, wherein said therapeutic composition is formulated as a delayed enteric release form.

15. (canceled)

16. (canceled)

17. The method of claim 1, wherein said therapeutic composition is in a liquid, frozen, freeze-dried, spray-dried, foam-dried, or powder form.

18. The method of claim 1, wherein said therapeutic composition comprises a cryoprotectant.

19. The method of claim 1, wherein said subject is pretreated with an antibiotic prior to administration of said composition.

20. The method of claim 1, wherein said method eliminates or reduces one or more chronic fatigue syndrome symptoms selected from the group consisting of post-exertion malaise lasting more than 24 hours; unrefreshing sleep; sleep deprivation; lethargy or fatigue; significant impairment of short-term memory or concentration; muscle pain; multi-joint pain without swelling or redness; headaches of a new type, pattern, or severity; tender cervical or axillary lymph nodes; a sore throat that is frequent or recurring.

21. The method of claim 1, wherein said administering further treats at least one additional disorder in said patient, wherein said disorder is selected from the group consisting of: a gastrointestinal dysbiosis, diarrhea, abdominal discomfort, bloating, flatulence and depression.

22. The method of claim 21, wherein said disorder is diarrhea.

23. A method for treating chronic fatigue syndrome in a human subject in need thereof, said method comprising administering to said human subject a pharmaceutically active dose of a therapeutic composition comprising viable non-pathogenic and non-selected bacteria of a fecal microbiota derived from a stool sample of a healthy human donor.

24. The method of claim 23, wherein the composition is orally administered.

25. The method of claim 23, wherein said composition comprises a substantially complete fecal microbiota of said stool sample.

26. The method of claim 23, wherein said method comprises a first dosing schedule followed by a second dosing schedule, wherein said second dosing schedule comprises a maintenance dose lower or equal to the dose of said first dosing schedule.

27. The method of claim 23, wherein said administering further treats at least one additional disorder in said patient, wherein said disorder is selected from the group consisting of: a gastrointestinal dysbiosis, diarrhea, abdominal discomfort, bloating, flatulence and depression.

28. The method of claim 27, wherein said disorder is diarrhea.
